(19) United States
(12) Patent Application Publication
(10) Pub. No.: US 2008/0161708 A1
(43) Pub. Date: Jul. 3, 2008

(54) Detecting Prolonged Myocardial Repolarization Indicative of Cardiac Condition

(76) Inventors: David N. Kenigsberg, Southfield, MI (US); Sanjaya Khanal, Farmington Hills, MI (US); Marcin Kowalski, Detroit, MI (US); Subramaniam Krishnan, West Bloomfield, MI (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/588,585

(22) PCT Filed: Feb. 11, 2005
(86) PCT No.: PCT/US2005/004291

§ 371 Date: Aug. 4, 2006

Related U.S. Application Data
(60) Provisional application No. 60/544,171, filed on Feb. 11, 2004.

(51) Int. Cl. A61B 5/0468

(52) U.S. Cl. 600/516

(57) ABSTRACT

The present invention comprises methods and apparatus for detecting prolonged myocardial repolarization as an indicator of cardiac conditions, including without limitation, transmural ischemia. In some embodiments, without limitation, the present invention comprises methods and apparatus to detect prolonged repolarization using electrocardiographic and electrophysiological tools and measurements.

### CROSS-REFERENCE TO RELATED APPLICATIONS

This application claims priority based on U.S. Provisional Patent Application No. 60/544,171, filed Feb. 11, 2004, which is hereby incorporated by reference in full.

### FIELD OF THE INVENTION

The present invention relates to methods and apparatus for detecting prolonged myocardial repolarization as an indicator of cardiac condition, including without limitation, transmural ischemia. In some embodiments, without limitation, the present invention comprises methods and apparatus to detect prolonged repolarization using electrocardiographic and electrophysiological tools and measurements.

### BACKGROUND

Many cardiac conditions can be detected by measuring and recording electrical signals from the heart. When displayed, the electrical signals form various patterns, which can be visually recognized. Electrocardiograms (“ECGs”) are routinely used for diagnosis and therapy of various cardiac conditions. However, the electrocardiograms measured and used in the usually described format have significant limitations in detecting and identifying certain cardiac conditions, as one example only, cardiac ischemia.

Analysis of cardiac signals, which is routinely performed in electrocardiography, is generally based on visual inspection to quantify or qualify signal morphology for the purpose of identifying and classifying abnormal patterns. Certain morphological characteristics of commonly recorded signals have high diagnostic value. The morphology and interval times recorded in the electrocardiogram generally provide a wealth of information about the state of the heart. Accordingly, automated approaches for identifying and classifying abnormalities in signals, such as cardiac signals, have been sought for use in determining a signal's morphologic characteristics.

However, given the wide diversity of possible shapes for cardiac signals, it is usually not possible for an automatic approach to identify significant characteristics that can be used for unambiguous classification. Rather, automated classification approaches generally compare the entire morphological shape of a signal with the shape of similar signals with known abnormalities but without particular regard to the specific characteristics that the signals contain. Alternatively, automated classification approaches restrict the automated examination only to those signals that are essentially normal and use detailed metrics (for example QRS width, QT interval or ST segment amplitude) of the essentially normal morphology for classifying abnormalities.

Despite its importance in the analysis of biologic signals, the automated and accurate identification and quantification of the significant morphological characteristics (for example, turns, peaks, knees, inflection points, and the like) in any cardiac signal (both abnormal as well as normal) are still in a developing stage. Existing methods have used the concept of sharpness (for example to detect R-waves) but have had limited success. This is due in part to the overly simplistic mathematical treatment this concept has received, as reflected in the rudimentary algorithms used for these measurements. Most of the current detection methods rely on three point interpolations to measure sharpness.

The simplest and most commonly used methods for measuring peaks of R-waves are based upon Taylor-series approximations to estimate the second derivative of the sensed signal. The formula utilizes highly local information (the point at the peak and its two close neighbors) ignoring nearby points that may contribute to signal peak. Other popular approaches utilize less local data, such as the peak and two adjacent extremes. All of these methods, which rely on three-point estimates of sharpness, may produce inaccurate estimates if waveforms are complex or are contaminated with noise. Thus, a need exists for automated identification and classification of peaks, knees, inflection points, and the like in sensed cardiac signals that takes into account wave scale and complexity that can yield a more accurate estimate of peaks for identifying and classifying abnormalities in cardiac signals.

Transmural ischemia is currently identified electrocardiographically by analyzing the QRS, ST and T-waves morphology. However, the changes in these electrocardiographic signals are neither very sensitive nor specific in detecting transmural ischemia. Waveforms are often not able to detect the occurrence of transmural ischemia because depending upon the timing of the ECG, classical changes, such as ST segment elevation or Q waves, may be absent. As a result, many individuals may be experiencing transmural ischemia but are never diagnosed. There are nearly one million people in the United States alone who develop an acute MI and who might benefit for improved detection and treatment. It would therefore be beneficial to develop a method and device for detecting the occurrence of transmural ischemia by measuring the duration of myocardial repolarization.

### SUMMARY OF THE INVENTION

The present invention was developed in light of these and other drawbacks and the unmet need in the art.

Generally, the present invention comprises methods and apparatus for the detection of prolonged myocardial repolarization relating to cardiac conditions, as examples only, transmural ischemia/myocardial infarction.

Previously, it was known in the art that the metabolic changes that occur early on in ischemic hearts promote the abbreviation of the QT interval. In contradistinction, the methods and apparatus of the present invention detect the prolongation of myocardial repolarization, including without limitation, prolongation of the QT interval. Thus, it has been discovered unexpectedly by Applicants that prolongation of the QTc occurs prior to other changes during acute transmural ischemia. The prolongation of the QT interval is one of the first detectable symptoms of transmural ischemia.

There are presently significant limitations in diagnosis of transmural ischemia/myocardial infarction. There are no absolutely specific tests to diagnose acute transmural ischemia. Classic teaching describes that acute myocardial infarction is usually manifested on the electrocardiogram by ST elevation followed by the development of Q waves (Braunweld et al.) However, this is not seen in all cases of transmural ischemia, and often the only manifestations are non-specific signs such as ST depression or T wave inversions. Therefore, while evaluating a patient for a suspected acute coronary syndrome, often more importance is given to clinical symptoms suggestive of myocardial ischemia (chest pain, diaphoresis, dyspnea, nausea, etc.). Certain patient populations lack these clinical features. Silent ischemia is known to occur in diabetics, women and the elderly (Epstein et al.) Also, patients who are status post orthotopic cardiac transplant, a rapidly increasing patient population, are at increased risk for myocardial infarction, however are denervated and hence do not present with chest pain. It is therefore not surprising that a significant percentage of patients with acute coronary syndromes presenting to the emergency room are often discharged without making the diagnosis.

The cyclical nature of thrombosis is well recognized. Articles by DeWood et al. show that during a myocardial infarction, there is a cyclical pattern of coronary artery occlusion, thrombolysis, and reocclusion. Therefore, depending on the period of time the electrocardiogram is recorded, the classical changes (ST segment elevation or Q waves) may be absent.

To meet the unmet need in the art, we have discovered novel methods and apparatus to detect prolonged myocardial repolarization and therefore to diagnose cardiac conditions, including without limitation, acute transmural ischemia and infarction. We have discovered in our studies that prolongation of the QTc occurs before other changes consistently (100% of the time) and predictably during acute transmural ischemia caused by balloon occlusion of the coronary artery, and subsequently normalizes after restoration of flow. This is a novel and unexpected finding because the metabolic changes that occur in ischemic heart muscle promote shortening of repolarization (Krishnan et al.) Based on our studies, this finding is a more reliable and applicable method to detect acute transmural ischemia.

We have studied ECG changes in patients in whom transmural ischemia was created during balloon angioplasty. We have also confirmed this in a controlled setting during coronary occlusion in anesthetized dogs. To our knowledge, the prolongation of QTc in this setting has never been studied or described. As far as we are aware, there have been no publications or description in the literature about QTc prolongation during acute coronary occlusion. Our findings are supported by research performed in ischemic porcine myocardium which shows that the action potential (the cellular equivalent of the QT interval) from the epicardium prolongs during the first two minutes of ischemia (Watanabe et al.)

The present invention may greatly enhance the diagnosis and therapy of brief or intermittent episodes of transmural ischemia. If the patient's baseline ECG and/or QTc is available, measurement of QTc may provide an extremely sensitive and specific way to detect transmural ischemia very early in its course. From our discovery. QTc prolongation in this setting should be very sensitive to detect early transmural ischemia.

Our findings support the development of embodiments of the invention comprising, without limitation, portable/wearable ECG machines capable of monitoring the QT interval to detect ischemia. In some specific embodiments, without limitation, the present invention may comprise a device for detecting transmural ischemia/myocardial infarction by analyzing electrocardiograms. In such embodiments, the device may comprise any device, whether automated, manual, or a combination of each, which is capable of reading an ECG. The device would read the ECG and detect prolongation of the QT interval by comparison with patient baseline data and/or reference data.

More specifically, the device of the present invention would function by reviewing the ECG. Then the device would quantitate the results of the EGG and compares the results with a standard to determine if there is deviation from the standard. Specifically, if the device detects a prolongation of the QT interval, it would be indicative of transmural ischemia.

Preferably, the device of the present invention would comprise an algorithm developed to analyze the results of an EGG by comparing patient baseline ECG and/or reference data with ECG data at the time of clinical evaluation. The algorithm would analyze the results of the later EGG and determine whether or not a pathologic coronary event, such as an occlusion, has occurred. Thus, the methods and devices of the present invention may detect transmural ischemia caused by myocardial failure and complications thereof including, but not limited to, arrhythmia, myocardial infarction, and myocardial failure by detecting the prolongation of the QT interval.

The invention has been described in an illustrative manner, and it is to be understood that the terminology that has been used is intended to be in the nature of words of description rather than of limitation. Other aspects of the invention will become apparent to those skilled in the art after reviewing the drawings and the detailed description below.

### BRIEF DESCRIPTION OF THE DRAWINGS

The present invention will now be described, by way of example, with reference to the accompanying drawings, in which:

FIGS. 1(A) and 1(B) are charts showing QT prolongation during transmural ischemia with a single beat in lead V2 at baseline (FIG. 1(A)) and during balloon inflation (FIG. 1(B)).

FIG. 2 is a graphical representation study data showing corrected QT interval increase.

FIGS. 3(A) through (C) are charts showing an activation recovery interval from the unipolar electrograms.

FIG. 4 is a graphical representation of ischemic preconditioning showing the decrement in prolongation of the corrected QT interval (QTc) with serial inflations during PCI.

FIGS. 5(A)-(C) shows an intracoronary electrogram.

FIGS. 6(A)-(B) is a chart showing one example, without limitation, of lead placement during percutaneous coronary intervention for surface ECG acquisition.

FIG. 7 is a picture of one embodiment, without limitation, of a device to measure intracoronary electrograms comprising a standard angioplasty wire and an ECG machine.

### DETAILED DESCRIPTION

Generally, the present invention comprises methods and apparatus for the detection of prolonged myocardial repolarization. In some preferred embodiments, the invention can be used to diagnose even brief episodes of transmural ischemia (<2 minutes). More specifically, some embodiments of the invention may comprise a device to measure the duration of myocardial repolarization during transmural ischemia/infarction.

The normal ECG is composed of a P wave, a QRS complex and a T wave. Generally the P wave represents atrial depolarization, the QRS complex represents ventricular depolarization, and the T wave reflects the phase of rapid repolarization of the ventricles. This configuration can be represented as follows:

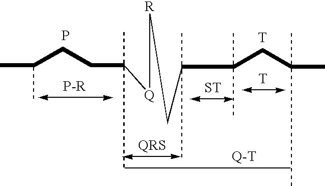

One of ordinary skill in the art will appreciate that detection and measurement of changes and abnormalities in this ECG wave may be indicative of cardiac condition, including without limitation, cardiac pathologies.

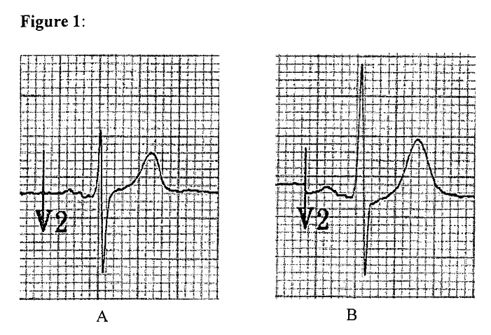

Without limiting the scope of the invention, in some embodiments, the invention may comprise a device, whether automated, manual, or combinations of both, which can accurately measure the QT interval and other indices of measuring the duration or myocardial repolarization. Classical cardiology and physiology teachings state that the metabolic changes that occur in ischemic hearts promote the abbreviation of the myocardial repolarization and the QT interval. Unexpectedly to the contrary, the present invention detects the prolongation of the QT interval and of the duration of myocardial repolarization as a marker of transmural ischemia. (See, e.g., FIGS. 1(A) and 1(B), showing QT prolongation during transmural ischemia with a single beat in lead V2 at baseline (FIG. 1(A)) and during balloon inflation (FIG. 1(B)).

Thus, in accordance with the invention, Applicants have unexpectedly discovered that prolongation of the QTc and duration of myocardial repolarization occur prior to other changes during acute transmural ischemia. Thus, prolongation of the QT interval is one of the first detectable symptoms of transmural ischemia, a consistent finding among studied human patients.

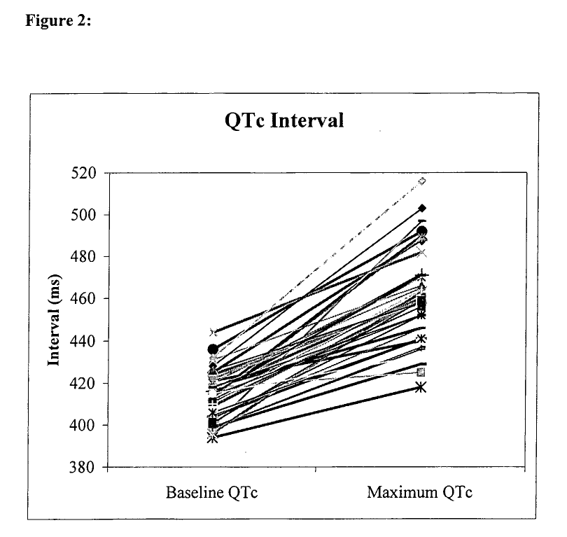

In our studies, the corrected QT interval on the surface electrocardiogram is the earliest and most consistent (e.g., 100%) finding during transmural ischemia induced by balloon occlusion during percutaneous coronary angioplasty (“PCA”). (See e.g., FIG. 2, a graphical representation of study data showing corrected QT interval increase from baseline with early transmural ischemia, occurring universally (100% of the time)). This should be contrasted with the fact that in our studies, substantially fewer numbers of patients developed chest pain (33%) or ST segment elevation (50%), the classical electrocardiographic feature of transmural myocardial ischemia.

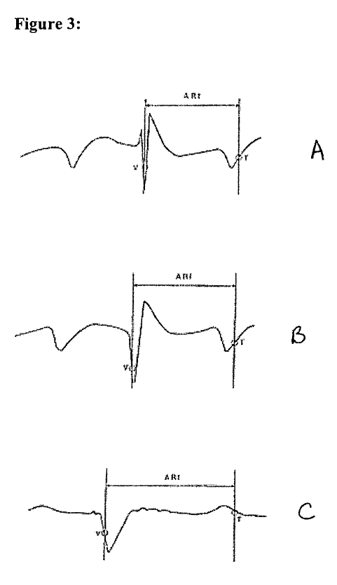

The QT interval is one measure of repolarization of the ventricular electrical signal on the surface electrocardiogram. In addition, some embodiments of the invention may comprise other methods to assess prolonged repolarization, including without limitation, intracardiac electrograms, assessing activation recovery intervals (“ARI”), and monophasic action potentials. As some examples, without limitation, FIGS. 3(A) through (C) are charts showing an activation recovery interval from the unipolar electrograms. The dV/dTₘₐₓ was used to define LRT for negative (FIG. 3(A)) and biphasic (FIG. 3(B)) T waves. For positive T waves, the dV/dTₘᵢₙ on the descending limb of the T wave was used (FIG. 3(C)). ARI was calculated at each site as the difference between LRT and LAT (determined from the dV/dTₘᵢₙ in the local QRS). (Figure adapted from L. Gepstein, G. Hayam, and S. A. Ben-Haim, Activation-Repolarization Coupling in the Normal Swine Endocardium, Circulation, Dec. 2, 1997; 96(11): 4036-4043.)

In accordance with some embodiments, unipolar electrograms from ventricular epicardium can be analyzed for the timing of local excitation and repolarization. The most rapid decrease in voltage in the QRS (dV/dt min) is the local excitation time, and the maximum rate of voltage increase (dV/dt max) near the peak of the T wave is local repolarization time. The difference between dV/dt min and dV/dt max is the ARI. ARI is related to the net effect of the durations of the action potentials at that site. Prolongation in ARI, as a measure of prolongation of local repolarization time, represents transmural ischemia, in accordance with the invention.

Monophasic action potentials (MAPs) are extracellularly recorded wave forms that reproduce the repolarization time course of transmembrane action potentials. (See, e.g., Franz M R, Current status of monophasic action potential recording: theories, measurements and interpretations, Cardiovasc Res. 1999; 41:25-40.) These MAPs can be measured in the in situ beating heart, in human subjects, by pressing a nonpolarizable electrode up against the endocardium or epicardium. Prolongation in MAP, as a measure of prolongation of repolarization time, represents transmural ischemia, in accordance with the invention.

During a myocardial infarction, intermittent occlusion can occur, resulting in cyclical flow, resulting in the absence of classical ECG changes. In accordance with the invention, without limitation, some embodiments may comprise technology incorporated into any external (wearable monitor), internal (implantable), or intracardiac devices (using QT/QTc intervals, activation recovery intervals, monophasic action potential durations) may be used to accurately measure repolarization times and thus enhance the sensitivity in detecting transmural myocardial ischemia.

In some embodiments, the present invention would comprise a device that functions by reviewing the QT interval of the ECG and various indices of myocardial repolarization. The device would quantitate the QT interval or duration of myocardial repolarization and compare the results with a standard (as only some examples, patient baseline data and/or reference data) to determine if there is deviation from the standard and tracks changes over time. Specifically, if the device detects a prolongation of the QT interval, it would be indicative of transmural ischemia.

The algorithm of some embodiments of the invention could be included in a portable electronic device that is worn by an individual. The device could be worn by an individual in order to detect ischemia or other cardiac conditions. Alternatively, the device could be located at a hospital or doctor's office. It could also be incorporated into a device implanted in the body. The device could be used to detect ischemia in patient's presenting with or without symptoms. It could also be used in monitoring high risk patients for early transmural ischemia.

We have also studied quantification of the amount of myocardium at risk and demonstrated a close correlation between the degree of QT prolongation and the percentage of myocardium at risk during brief episodes of transmural ischemia. The degree of QT change detected in accordance with the invention can be used to identify the amount of myocardium at risk. Where the area of myocardium at risk is small, the QT prolongation will be less than where the area of myocardium at risk is larger. Thus, in accordance with some embodiments, the invention can be utilized during percutaneous or surgical cardiac procedures to estimate the amount of myocardium at risk distal to the coronary artery occlusion.

During percutaneous coronary intervention (“PCI”), despite the fact that a coronary stenosis is deemed significant (>75% occluded) as estimated by angiographic luminal diameter, the clinical significance in intervening on that stenosis is not predictable. After one balloon inflation, it can be determined, using an increase in QT as a guide, whether or not the tissue downstream from the occlusion is viable or hibernating. If there is no demonstrable increase in QT prolongation with balloon occlusion, it can be assumed that the tissue in question is necrotic or dead myocardium. If the latter is the case, abortion of the procedure may be indicated which would save the operator time and effort and the patient from potential risk of the futile procedure. A device like some embodiments of the present invention that can measure changes in QT distal to the occlusion would serve in this situation. This device can be used intracoronary or transcutaneously, as one example only, a wire with ability to conduct electricity can be used to measure and estimate myocardial viability. QT prolongation as measured in the heart or from the body surface can serve this function as well.

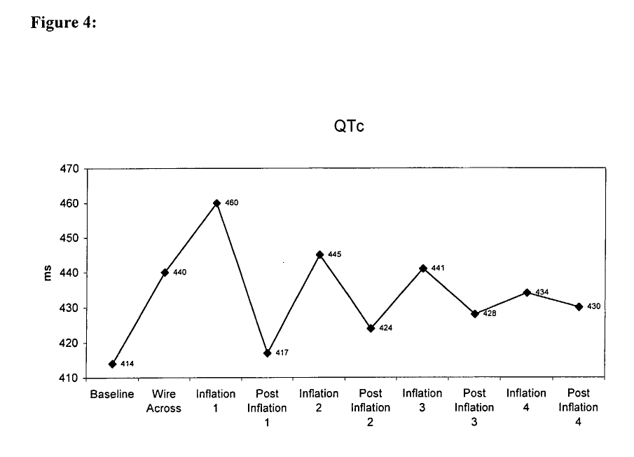

In other embodiments, the invention comprises methods and apparatus directed to monitoring myocardial preconditioning. QT prolongation occurs with each balloon inflation, however, there is a decrement seen during successive inflations. (See, e.g., FIG. 4, showing the decrement in prolongation of the corrected QT interval (QTc) with serial inflations during PCI.) This decrement represents ischemic preconditioning. Ischemic preconditioning is a phenomenon whereby the heart muscle gets less ischemic with each subsequent similar duration cessation of blood flow. This phenomenon protects the heart from sequential ischemic episodes. In some embodiments, the present invention can detect ischemic preconditioning by measuring the change in myocardial repolarization, for example, by permitting the detection of decrements in QT prolongation. This would be very helpful in identifying new therapies for the heart. In an animal model, therapies, including without limitations, drug therapies, that promote ischemic preconditioning could be tested using embodiments of the present invention. Since a decrement in QT prolongation represents ischemic preconditioning, this can be used as a means to conduct further investigation and therapeutic discovery for the heart.

In other embodiments, without limitation, the invention comprises a method of measuring myocardial repolarization changes during ischemia. Change in myocardial repolarization from baseline is the earliest and most sensitive method of detecting myocardial ischemia. Thus, some embodiments of the invention would comprise an automated device that records, stores and reports the change in myocardial repolarization during ischemia. Some embodiments of the invention would comprise a device that can measure the change in duration of myocardial repolarization during ischemia in most or all clinical settings, for example and without limitation, with a Holter monitor of suitable frequency; telemetry-based monitor; during angioplasty and stenting; with implantable device (e.g., permanent pacemaker, internal cardiac defibrillator, hemodynamic recorder, etc.), and/ or surface device (e.g., ECG cart).

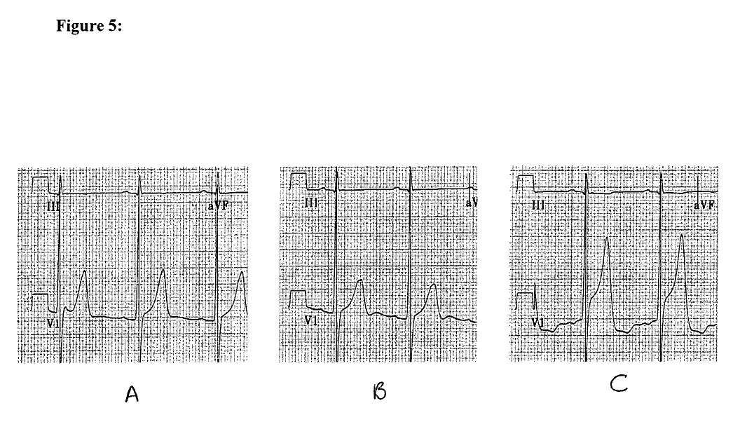

Other embodiments comprise methods and apparatus to diagnosis and monitor myocardium at risk. The percent change in the duration of myocardial repolarization can be used to identify the amount of myocardium at risk. Thus, in some embodiments, the invention comprises methods and devices that use an algorithm to detect, record, calculate and store the percent change in the duration of myocardium repolarization on a surface or by intracoronary electrocardiogram. (See e.g. FIGS. 5(A)-(C), an intracoronary electrogram (labeled V1) at baseline (FIG. 5(A)), during first balloon inflation (FIG. 4(B)) and during subsequent balloon inflation (FIG. 5(C)). Note the fact that QT prolongation occurs before ST segment elevation becomes apparent.)

Some embodiments comprise methods and apparatus directed to the diagnosis and monitoring of myocardial viability. Thus, in some embodiments, the invention can comprise a device that uses an algorithm to detect, record, calculate and store the percent change in the duration of myocardium repolarization with the intention to identify viable myocardium.

### EXAMPLES

By way of example, without limiting the scope of embodiments of the invention, the invention may comprise some or all of the following steps.

Some embodiments of the invention may comprise several steps of a method for determining prolonged myocardial repolarization by accumulation and analysis of patient data by comparison to patient baseline and/or reference data.

For example, after signing informed consent, patients are prepared for elective PCI in standard fashion. Patient history and demographic information, including for example, age, gender, height, weight, family history of premature cardiovascular disease, a past medical history of hypertension, hyperlipidemia, diabetes, CVA, renal failure, peripheral vascular disease, prior CABG, prior MI, prior PCI, prior or current tobacco use, current medications, or the like.

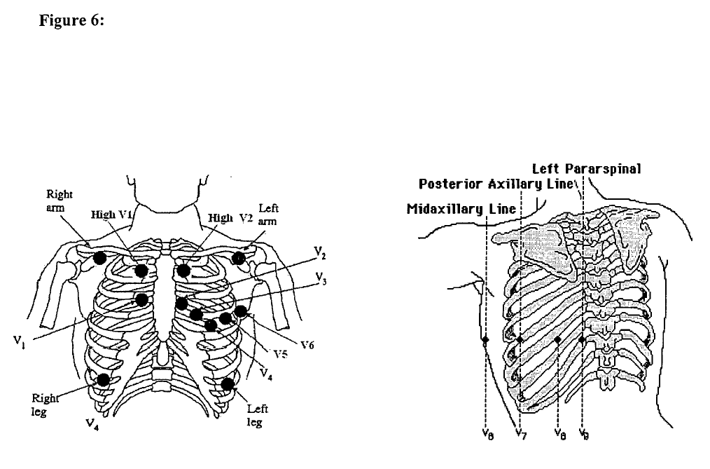

ECG electrodes are suitably placed on the subject. (See for example, FIGS. 6(A)-(B)). During the placement of electrodes for the surface electrocardiogram, standard limb leads and precordial leads are placed and connected to a MAC 8 Marquette ECG Cart. Three additional/auxiliary leads are placed as well and change based on the artery being intervened upon during the PCI: in Left Anterior Descending artery angioplasties these additional leads were High V1, High V2 and V9; in Circumflex artery angioplasties V7, V8 and V9; and in Right Coronary angioplasties V3R, V4R and High V1.

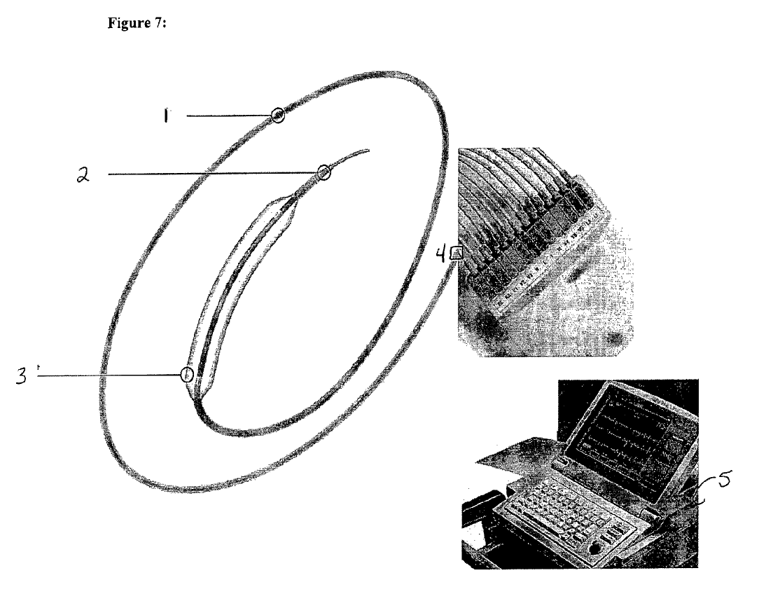

Intracoronary electrogram recording is conducted. A standard angioplasty wire is connected at its distal end to an alligator clip. The other end of the alligator clip is connected to the V1 lead of the ECG cart. (See e.g., FIG. 7, a representation of a device to measure intracoronary electrograms comprising a standard angioplasty wire and an ECG machine, including the proximal end (1) and distal end (2) of a guidewire, an angioplasty balloon (3), and the proximal end of the guide wire attached to the V1 lead (4) of an ECG machine (5)). ECG recording is performed. A baseline electrocardiogram is obtained before the procedure begins in order to establish the baseline QT in the patient. This is obtained before sedative drugs are administered.

The patient is sterilely draped and the procedure begins. The operators perform percutaneous angioplasty of the lesion in standard fashion and in accordance with their practice normally.

ECGs are obtained during inflation of the balloon at 19 second intervals until balloon deflation. Typical angioplasty requires at least one balloon inflation and may involve more than five inflations. Each inflation varies in length from 15 seconds to 60 seconds and is dependent on the operator preference and the clinical situation. During a 15 second inflation, one ECG can be obtained, during a 60 second inflation 3 ECGs.

ECGs are also obtained after each deflation and before the next inflation, if time permits. As the ECGs are obtained, they are printed and labeled. The baseline ECG is labeled “Baseline.” The ECGs obtained during each inflation is labeled with the inflation number and time from when the balloon inflation begins, the presence or absence of chest pain, the balloon type being used, the atmospheres of pressure used to inflate the balloon and the presence or absence of a stent. The post inflation ECGs are labeled according to which inflation they succeed.

After the ECGs are recorded, the ECG machine data is uploaded onto the MUSE system and digital records of each ECG are saved onto floppy diskettes.

ECG analysis is conducted. There are at least three different methods by which each ECG may be analyzed:
1. Hand measurement: A visual based measurement with calipers performed by a Board Certified electrophysiologist who is blinded to the labeling of the ECG;
2. MUSE System [GEHC, Menomonee Falls, Wis.] (MUSE): Automated analysis of QT which combines ECG complexes from 12 standard leads into 1 waveform beat and measures the QT from the onset of the Q-wave to the end of the T wave of the combined waveform; and/or
3. ECG Interval Editor [GEHC, Menomonee Falls, Wis.] measures the QT interval in each lead (from beginning of the earliest Q-wave to the end of the latest T-wave) and combines the measurements using the vector magnitude equation.

Each ECG is analyzed for ST segment elevation or depression, T wave inversion, QRS complex prolongation or abbreviation, and QT prolongation or abbreviation. The QTc interval is calculated using the Bazett correction method, which takes into account the heart rate. QT dispersion, the minimal QT subtracted from the maximal QT, is calculated.

Patient data is accumulated. Patient demographic, electrocardiographic and angiographic data are inputted into a spreadsheet. All of the ECGs are entered into the spreadsheet according to their label. All of the baseline ECGs are averaged together; the last ECGs of each inflation 1 are averaged together, the last post inflation 1 ECGs are averaged together, and so on and so forth. The data are analyzed to determine whether myocardial repolarization is prolonged in relation to patient baseline or standardized data. QT prolongation is defined as an increase in QT interval during balloon inflation as compared to the baseline ECG. The QT interval is measured in milliseconds.

By way of another example, without limitation, we determined unexpectedly that QT prolongation precedes ST segment elevation during balloon angioplasty in a thirty-seven patient population. In this example, we assessed the time to QT prolongation, onset of chest pain and presence of ST segment elevation (STE) or ST segment depression (STD) during PCI.

We prospectively analyzed serial electrocardiograms (ECG) obtained at 20 second (s) intervals in 37 patients undergoing elective PCI: 11 of the right coronary artery, 14 of the left circumflex artery and 12 of the left anterior descending artery. In each of the ECGs, we examined ST segment changes as well changes in the QT and QTc interval (QTc). We also obtained data regarding the presence or absence of chest pain during each ECG recording. 36 patients underwent 2 inflations, 22 underwent 3, and 12 underwent 4 inflations. The mean inflation times were 42.1 s, 51.3 s, 44.2 s, and 31.9 s during the 1st, 2nd, 3rd and 4th inflations, respectively. Only 17 of 37 patients had STE and 7 of 37 had STD, despite the use of additional ECG leads (15). In those patients with STE or STD, changes were seen during each inflation at 37+/−9 s, on average, and returned to baseline after the inflation. 16 of 31 patients had chest pain with balloon inflation which occurred after 22+/−4 s. Prolongation of the QT and QTc were seen in 100% of patients during each inflation at 16 +/−7 s, on average. QT and QTc returned to baseline after each inflation. Thus, we discovered unexpectedly that prolongation of the QT is seen as the earliest ECG change in patients undergoing elective PCI. This occurs after the description of chest pain, but precedes STE or STD. In another example, without limitation, in accordance with the invention, we evaluated ischemic preconditioning during percutaneous coronary intervention as manifested by QTc variation in an 37 patient population. Previously, no data existed regarding changes in QT (QT) and QTc intervals (QTc) during transmural ischemia caused by balloon occlusion and stenting of the coronary artery.

In our study, after obtaining informed consent from 37 patients undergoing elective balloon angioplasty and stenting, 15 lead electrocardiograms (ECGs) were recorded serially at baseline, at 20 second (s) intervals during each inflation, and after each inflation. Average QT and QTc was determined on each ECG and results were recorded.

We found that the baseline QTc was 414+/−14 ms. During balloon angioplasty, the mean QTc with the 1st inflation was 460+/−21 ms and returned to near-baseline after the inflation. Subsequent average QTc values were 445+/−20 ms with the 2nd inflation and 450+/−19 ms with the 3rd inflation, and 436+/−14 ms for the 4^{th} inflation. QTc prolongation increased further with stenting, mean QTc 460+/−23 ms.

In accordance with the invention, we discovered that there is decrement in QTc prolongation with repetitive transmural ischemic episodes induced by temporary coronary occlusion with balloon inflation during percutaneous coronary intervention suggestive of ischemic preconditioning. The further increment in QTc prolongation seen with stent deployment needs to be investigated.

In a third example, without limitation, we discovered unexpectedly that QT prolongation is the earliest and most consistent electrocardiographic change seen in studied patients during balloon occlusion of the coronary artery.

Prolongation of the QT interval (QT) has been described in patients with acute coronary syndromes, but not in patients undergoing percutaneous coronary intervention (PCI). In accordance with the invention, we prospectively recorded 15 lead ECGs at 20 second(s) intervals in 37 patients undergoing elective PCI: 11 right coronary artery (RCA), 14 left circumflex artery (LCx) and 12 left anterior descending artery (LAD). QT, QTc interval (QTc) and OT dispersion (OTd, maximal QT−minimal QT) were measured in each ECG. We analyzed the ECGs for the presence and time to QT prolongation, onset of chest pain and presence of ST segment elevation (STE), ST segment depression (STD) and T wave changes.

36 patients underwent 2 balloon inflations, 22 underwent 3, and 12 underwent 4 inflations. The mean inflation times were 42.1 s, 51.3 s, 44.2 s, and 31.9 s during the 1^{st}, 2^{nd}, 3^{rd} and 4^{th} inflations, respectively.

We discovered that prolongation of QT, QTc and QTd were seen in 37 out of 37 patients during each inflation at 16+/−7 s, with a return to baseline after the inflation. QT, QTc and QTd prolonged on average by 27 ms, 45 ms, and 11 ms for the 1^{st} inflation; 22 ms, 30 ms and 17 ms for the 2 inflation; 18 ms, 35 ms, 9 ms for the 3^{rd} inflation; and 25 ms, 21 ms and 15 ms for the 4^{th} inflation, respectively. STE, STD and T wave inversions were seen in 17.7 and 5 patients, respectively. These changes were seen during each inflation at 37+/−9 s, on average, and returned to baseline after the inflation. 16 of 31 patients had chest pain with balloon inflation which occurred after 22+/−4 s. Thus, prolongation of the QT, QTc, and QTd are the earliest and most consistent ECG changes during early transmural ischemia.

In another example, without limitation, we discovered unexpectedly that QT prolongation is the most consistent electrocardiographic change seen in studied patients during balloon occlusion of the coronary artery with percutaneous coronary intervention.

Prolongation of the QT interval (QT) has been described in patients with acute coronary syndromes, but not in patients undergoing percutaneous coronary intervention (PCI). We prospectively evaluated the presence of QT prolongation during PCI.

We recorded 15 lead ECGs at 20 second(s) intervals in patients undergoing elective PCI: 6 right coronary artery (RCA), 6 left circumflex artery (LCx) and 3 left anterior descending artery (LAD). In each ECG, we examined ST segment and T wave changes, QT, QTc interval (QTc) and QT dispersion (QTd, maximal QT−minimal QT).

In our study, 14 patients underwent 2 inflations, 9 underwent 3, and 7 underwent 4 inflations. The mean inflation times were 50 s, 57 s, 48 s, and 33 s during the 1^{st}, 2^{nd}, 3^{rd} and 4^{th} inflations, respectively. ST elevation, depression and T wave inversions were seen in 6, 2 and 3 patients, respectively. Prolongation of the QT, QTc and QTd were seen in all patients during each inflation, with a return to baseline after the inflation. QT, QTc and QTd prolonged on average by 24 ms, 37 ms, and 9 ms for the 1^{st} inflation; 22 ms, 31 ms and 16 ms for the 2^{nd} inflation; 7 ms, 16 ms, 6 ms for the 3^{rd} inflation; and 28 ms, 31 ms and 15 ms for the 4^{th} inflation, respectively. In LAD PCI, QT prolongation was seen in V1-V6. In LCx PCI, QT prolongation was seen in II, III, aVF and V7-V9. In RCA PCI, it was seen in II, III, aVF, v3R and v4R.

| | Baseline | Peak | P value |
|---|---|---|---|
| Mean QT (ms) | 400 +/− 43.6 | 436.7 +/− 46.7 | <.0001 |
| Mean QTc (ms) | 414.3 +/− 12.6 | 468 +/− 29.3 | <.0001 |
| Mean QTd (ms) | 85.4 +/− 33.3 | 131.5 +/− 31.8 | .0012 |

Thus, in accordance with the invention, we discovered in our studies that prolongation of the QT, QTc, and QTd is the most consistent (100%) changes during transmural ischemia induced by balloon inflation during PCI.

This application may reference various publications by author and/or by patent number, including United States patents. Full citations of these and/or other references are listed below. The disclosures of each of these references in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation. Obviously, many modifications and variations of the present invention are possible in light of the above teachings. It is, therefore, to be understood that within the scope of the described invention, the invention may be practiced otherwise than as specifically described.

While the present invention has been particularly shown and described with reference to the foregoing preferred and alternative embodiments, it should be understood by those skilled in the art that various alternatives to the embodiments of the invention described herein may be employed in practicing the invention without departing from the spirit and scope of the invention as defined in the following claims. It is intended that the following claims define the scope of the invention and that the method and apparatus within the scope of these claims and their equivalents be covered thereby. This description of the invention should be understood to include all novel and non-obvious combinations of elements described herein, and claims may be presented in this or a later application to any novel and non-obvious combination of these elements. The foregoing embodiments are illustrative, and no single feature or element is essential to all possible combinations that may be claimed in this or a later application. Where the claims recite “a” or “a first” element of the equivalent thereof, such claims should be understood to include incorporation of one or more such elements, neither requiring nor excluding two or more such elements.

### REFERENCES

Alderman et al. The angiographic definitions of the Bypass Angioplasty Revascularization Investigation. Coronary Artery Disease 1992, 3:1189-07.
Antzelevitch C., Sicouri S., Litovsky S. H., Lukas A., Krishnan S. C., Di Diego J. M., Gintant G. A. Liu D. W. Heterogeneity within the ventricular wall. Electrophysiology and pharmacology of epicardial, endocardial, and M cells. Circulation Research. 69(6):1427-49, December 1991.
Braunwald E, Zipes D P, Libby P. Heart Disease: A Textbook of Cardiovascular Medicine. 2001; 1135-1136,
DeWood M A, Stifter W F, Simpson C S, Spores J, Eugster G S, Judge T P, Hinnen M L Coronary arteriographic findings soon after non-Q-wave myocardial infarction. New England Journal of Medicine. 1986; 315(7):417-23.
DeWood M A, Spores J, Notske R, Mouser L T, Burroughs R, Golden M S, Lang H T. Prevalence of total coronary occlusion during the early hours of transmural myocardial infarction. New England Journal of Medicine, 1980; 303(16):897-902.
Epstein S E, Quyyumi A A, Bonow R O. Myocardial Ischemia—silent or asymptomatic. New England Journal of Medicine. 1988; 318:1038.
Haws C W, Lux R L. Correlation between in vivo transmembrane action potential durations and activation-recovery intervals from electrograms: effects of interventions that alter repolarization time. Circulation. 1990; 81:281-288.
Krishnan S. C., Antzelevitch C. Sodium channel block produces opposite electrophysiological effects in canine ventricular epicardium and endocardium. Circulation Research. 69(2):277-91, August 1991.
Krishnan S. C., Josephson M. E. ST segment elevation induced by class IC antiarrhythmic agents: underlying electrophysiologic mechanisms and insights into drug-induced proarrhythmia. Journal of Cardiovascular Electrophysiology. 9(11):1167-72, November 1998.
Krishnan S. C., Shivkumar K., Garan H. Ruskin J. N. Increased vulnerability of the subendocardium to ischaemic injury: an electrophysiological explanation. Lancet. 346(8990):1612-4, Dec. 16, 1995.
Millar C K, Kralios F A, Lux R L. Correlation between refractory periods and activation-recovery intervals from electrograms: effects of rate and adrenergic interventions. Circulation. 1985; 72:1372-1379.
Wang K., Asinger R. W., Marriott H. J. L. ST-Segment Elevation in Conditions Other Than Acute Myocardial Infarction. N Engl J Med. 349:2128-2135, Nov. 27, 2003.
Watanabe I. Kanda A. Engle C L, Gettes L S. Comparison of the effects of regional ischemia and hyperkalemia on the membrane action potentials of the in situ pig heart. Journal of Cardiovascular Electrophysiology, 1997; 8 (11):1229-36.
Wyatt R. Comparison of estimates of activation and recovery times from bipolar and unipolar electrograms to in vivo transmembrane action potential duration. In: Proceedings IEE. Engineering Medical Biologists Society Second Annual Conference. Washington, D.C.; 1980:22.

### Publications

QT Prolongation is the Earliest and Most Consistent Electrocardiographic Change Seen in Patients During Balloon Occlusion of the Coronary Artery. D. Kenigsberg, S. Khanal, S. Krishnan. Europace. June, 2004: 6, Supplement 1, 167/4.
Ischemic Preconditioning During Percutaneous Coronary Intervention as Manifested by QTc Variation: A Thirty-Seven Patient Experience. D. Kenigsberg, S. Khanal, S. Krishnan. Europace. June, 2004: 6, Supplement 1, 98P/14.
QT Prolongation is the Most Consistent Electrocardiographic Change Seen in Patients During Balloon Occlusion of the Coronary Artery With Percutaneous Coronary Intervention. David N. Kenigsberg, Sanjaya Khanal, Subramaniam Krishnan. Journal of the American College of Cardiology. March, 2004: 43, Number 5, Supplement A, 130A.

### Presentations

QT Prolongation is the Earliest and Most Consistent Electrocardiographic Change Seen in Patients During Balloon Occlusion of the Coronary Artery. D. Kenigsberg, MD, S. Khanal, MD and S. Krishnan, MD. Henry Ford Hospital, Detroit, Mich., USA.
Poster, Michigan Chapter of the American College of Cardiology Annual Meeting, Traverse City, Mich., Oct. 15-16, 2004.
QT Prolongation is the Earliest and Most Consistent Electrocardiographic Change Seen in Patients During Balloon Occlusion of the Coronary Artery. D. Kenigsberg, MD, S. Khanal, MD and S. Krishnan, MD. Henry Ford Hospital, Detroit, Mich., USA. Oral Presentation, Cardiostim, Nice, France, Jun. 18, 2004.
Ischemic Preconditioning During Percutaneous Coronary Intervention as Manifested by QTc Variation: A Thirty-Seven Patient Experience. D. Kenigsberg, MD, S. Khanal, MD and S. Krishnan, MD. Henry Ford Hospital, Detroit, Mich., USA. Poster, Cardiostim, Nice, France, Jun. 17, 2004.
QT Prolongation is the Most Consistent Electrocardiographic Change Seen in Patients During Balloon Occlusion of the Coronary Artery With Percutaneous Coronary Intervention. David N. Kenigsberg, Sanjaya Khanal, Subramaniam Krishnan. Poster, ACC Annual Scientific Sessions, New Orleans, La., Mar. 8, 2004.
QT Prolongation is the Most Consistent Electrocardiographic Change Seen in Patients During Balloon Occlusion of the Coronary Artery With Percutaneous Coronary Intervention. David N. Kenigsberg, Sanjaya Khanal, Subramaniam Krishnan. Poster, First Annual Cardiology Fellowship Conference, New Orleans, La., Mar. 5, 2004.

What is claimed is:

1. A method for detecting prolonged myocardial repolarization as an indicator of transmural ischemia or infarction in a mammalian subject, comprising the steps of:
obtaining baseline electrocardiogram data derived from the subject;
performing an electrocardiogram on the subject after the baseline data was derived from the subject; and
comparing the baseline electrocardiogram data with data from the electrocardiogram to determine whether the time interval of myocardial repolarization is increased.

2. The method of claim 1, wherein the comparing step comprises comparing QT interval data in the baseline data with QT interval data from the electrocardiogram.

3. The method of claim 1, wherein the comparing step is automated in whole or in part.

4. The method of claim 1, wherein the comparing step is manual in whole or in part.

5. The method of claim 1, wherein the subject is a human.

6. The method of claim 5, wherein the human subject is suspected of having suffered myocardial ischemia after the baseline electrocardiogram data was derived from the subject.

7. A method for detecting prolonged myocardial repolarization as an indicator of transmural ischemia or infarction in a mammalian subject, comprising the steps of:
obtaining baseline cardiac electrical data derived from the subject;
measuring cardiac electrical activity in the subject after the baseline data was derived from the subject; and
comparing the baseline data with data from the measurement of the cardiac electrical activity to determine whether the time interval of myocardial repolarization is increased.

8. The method of claim 7, wherein the comparing step comprises comparing activation recovery interval data in the baseline data with activation recovery interval data from the measurement of cardiac electrical activity.

9. The method of claim 7, wherein the comparing step comprises comparing monophasic action potential data in the baseline data with monophasic action potential data from the measurement of cardiac electrical activity.

10. A method of detecting myocardium at risk or myocardial viability by measurement of changes in myocardial repolarization in a mammalian subject, comprising the steps of:
performing an electrocardiogram on the subject to obtain baseline electrocardiogram data;
performing a clinical procedure on the subject;
monitoring the subject's electrocardiogram during the clinical procedure to obtain electrocardiogram data; and
comparing the baseline electrocardiogram data with the electrocardiogram data from the clinical procedure to determine whether the time interval of myocardial repolarization is changed during the procedure.

11. The method of claim 10, wherein the subject is a human.

12. A method of detecting myocardium at risk or myocardial viability by measurement of changes in myocardial repolarization in a mammalian subject, comprising the steps of:
measuring baseline cardiac electrical data in the subject;
performing a clinical procedure on the subject;
measuring cardiac electrical data in the subject during the clinical procedure; and
comparing the baseline data with the cardiac electrical data from the clinical procedure to determine whether the time interval of myocardial repolarization is changed during the procedure.

13. The method of claim 12, wherein the subject is a human.

14. The method of claim 12, wherein the comparing step comprises comparing activation recovery interval data in the baseline data with activation recovery interval data from the measurement of cardiac electrical activity.

15. The method of claim 12, wherein the comparing step comprises comparing monophasic action potential data in the baseline data with monophasic action potential data from the measurement of cardiac electrical activity.

16. A method of detecting ischemic preconditioning by measurement of changes in myocardial repolarization in a mammalian subject, comprising steps of:
initiating an electrocardiogram on the subject;
performing a first step of a clinical procedure on the subject;
capturing first electrocardiogram data from the subject during the time period in which the first step of the clinical procedure was performed;
performing one or more additional steps of a clinical procedure on the subject;
capturing electrocardiogram data from the one or more additional steps of a clinical procedure; and
comparing the first electrocardiogram data with electrocardiogram data from the one or more additional steps to determine whether the time interval of myocardial repolarization is changed between the first step of the clinical procedure and any of the subsequent steps.

17. The method of claim 16, wherein the subject is a human.

18. A method of detecting ischemic preconditioning by measurement of changes in myocardial repolarization in a mammalian subject, comprising steps of:
initiating the measurement of cardiac electrical data in the subject;
performing a first step of a clinical procedure on the subject;
capturing cardiac electrical data from the subject during the time period in which the first step of the clinical procedure was performed;
performing one or more additional steps of a clinical procedure on the subject;
capturing cardiac electrical data from the one or more additional steps of a clinical procedure; and
comparing the first cardiac electrical data with cardiac electrical data from the one or more additional steps to determine whether the time interval of myocardial repolarization is changed between the first step of the clinical procedure and any of the subsequent steps.

19. The method of claim 18, wherein the subject is a human.

20. The method of claim 18, wherein the comparing step comprises comparing activation recovery interval data in the baseline data with activation recovery interval data from the measurement of cardiac electrical activity.

21. The method of claim 18, wherein the comparing step comprises comparing monophasic action potential data in the baseline data with monophasic action potential data from the measurement of cardiac electrical activity.

22. An electronic apparatus for detecting prolonged myocardial repolarization as an indicator of transmural ischemia or infarction in a mammalian subject, comprising:
Means for storing baseline electrocardiogram data derived from the subject;
Means for performing an electrocardiogram on the subject after the baseline data was derived from the subject; and
Means for comparing the baseline electrocardiogram data with data from the electrocardiogram to determine whether the time interval of myocardial repolarization is increased.

23. The apparatus of claim 22, wherein the subject is a human.

24. The apparatus of claim 22, wherein the comparing means comprises an algorithm.

25. An electronic apparatus for detecting prolonged myocardial repolarization as an indicator of transmural ischemia or infarction in a mammalian subject, comprising:
Means for storing baseline cardiac electrical data derived from the subject;
Means for measuring cardiac electrical data in the subject after the baseline data was derived from the subject; and
Means for comparing the baseline data with data from the measurement of cardiac electrical data from the subject to determine whether the time interval of myocardial repolarization is increased.

26. The apparatus of claim 25, wherein the subject is a human.

27. The apparatus of claim 25, wherein the comparing means comprises an algorithm.

28. The apparatus of claim 25, wherein the comparing means compares activation recovery interval data in the baseline data with activation recovery interval data from the measurement of cardiac electrical activity.

29. The method of claim 25, wherein the comparing means compares monophasic action potential data in the baseline data with monophasic action potential data from the measurement of cardiac electrical activity.

30. An electronic apparatus for detecting prolonged myocardial repolarization as an indicator of myocardium at risk or myocardial viability in a mammalian subject, comprising:
Means for storing baseline cardiac electrical data derived from the subject;
Means for measuring cardiac electrical data in the subject after the baseline data was derived from the subject; and
Means for comparing the baseline data with data from the measurement of cardiac electrical data from the subject to determine whether the time interval of myocardial repolarization is increased.

31. The apparatus of claim 30, wherein the subject is a human.

32. The apparatus of claim 30, wherein the comparing means comprises an algorithm.

33. The apparatus of claim 30, wherein the comparing means compares activation recovery interval data in the baseline data with activation recovery interval data from the measurement of cardiac electrical activity.

34. The method of claim 30, wherein the comparing means compares monophasic action potential data in the baseline data with monophasic action potential data from the measurement of cardiac electrical activity.

35. An electronic apparatus for detecting prolonged myocardial repolarization as an indicator of ischemic preconditioning in a mammalian subject, comprising:
Means for storing baseline cardiac electrical data derived from the subject;
Means for measuring cardiac electrical data in the subject after the baseline data was derived from the subject; and
Means for comparing the baseline data with data from the measurement of cardiac electrical data from the subject to determine whether the time interval of myocardial repolarization is increased.

36. The apparatus of claim 35, wherein the subject is a human.

37. The apparatus of claim 35, wherein the comparing means comprises an algorithm.

38. The apparatus of claim 35, wherein the comparing means compares activation recovery interval data in the baseline data with activation recovery interval data from the measurement of cardiac electrical activity.

39. The method of claim 35, wherein the comparing means compares monophasic action potential data in the baseline data with monophasic action potential data from the measurement of cardiac electrical activity.
